(19) United States
(12) Patent Application Publication
(10) Pub. No.: US 2021/0307439 A1
(43) Pub. Date: Oct. 7, 2021

(54) Easy-Fitting Coif for Scalp-Cooling Cap

(71) Applicant: Hair Clinic Reve-21 Corporation, Osaka (JP)

(72) Inventor: Katsumasa Okamura, Osaka-shi (JP)

(73) Assignee: Hair Clinic Reve-21 Corporation, Osaka (JP)

(21) Appl. No.: 17/330,406

(22) Filed: May 25, 2021

Related U.S. Application Data
(63) Continuation-in-part of application No. 16/461,790, filed on May 16, 2019.

(30) Foreign Application Priority Data
Nov. 17, 2016 (JP) 2016-005534

(51) Int. Cl. A42B 1/008

(52) U.S. Cl. CPC A42B 1/008

(57) ABSTRACT

Easy-fitting chemotherapeutic coif worn under, and configured to function in association with, the cooling-fluid circulating (heat-exchanging) cap of a scalp-cooling device or system. Finlike protrusions from left and right temporal pieces of the coif are furnished with hook-and-loop fasteners that oppose counterpart hook-and-loop fasteners furnished on left and right sides of the outer surface of a nape-covering section of the coif. Fitting the heat-exchanging cap over the coif on a wearer's head connects the opposing the hook-and-loop fasteners to fit the coif to the wearer's head dimensions, and firmly cover the nape. A handle is provided on an edge portion of a frontal section of the coif, and with the heat-exchanging cap in place may be tugged on by the wearer to pull out any wrinkles in the coif to eliminate any gap between the coif and the heat-exchanging cap.

This application is based upon, wherein the benefit of priority is claimed from, corresponding Japanese Patent Application No. 2016-005534, filed in the Japanese Patent Office on Nov. 17, 2016 and the entire contents of which are incorporated herein by reference.

### BACKGROUND OF THE INVENTION

### Technical Field

The present invention relates to headgear, in particular, to head coverings employed in therapeutic procedures involving the scalp.

### Description of the Related Art

Chemotherapies used in cancer treatment are often accompanied by hair loss, wherein patients' psychological distress and other significant lowering of quality-of-life has been a problem. It is known that such hair loss can be prevented or suppressed by keeping the scalp at a low temperature to keep the hair roots from absorbing the therapeutic drugs for chemotherapy. For this reason, head-cooling technology directed to preventing hair loss due to chemotherapy has been proposed—such as the device disclosed in Japanese Pat. No. 5,133,355—and is in commercial use in the medical industry—such as the system marketed by Paxman Coolers Limited/Paxman US, Inc. (“Paxman” hereinafter). The essential component of head-cooling devices of this kind is a head-conforming cap containing a cooling-medium passageway. A cooling medium is made to circulate in the cooling-medium passageway to cool the scalp by heat exchange between the scalp and the cooling medium. Technically, then, the cap is a heat exchanger.

The heat-exchanging cap—over which a head-enveloping cap retainer made of a soft fabric is worn—that is fitted onto the head of the user is often manufactured by molding from a synthetic resin such as a silicone rubber. Although silicone rubbers have a certain degree of flexibility, since the shape and size of users' heads vary, it is difficult to bring the heat-exchanging cap into close contact with the entire head. The inner surface of the heat-exchanging cap functions as a heat-exchange surface. If a gap arises between the user's scalp and the inner surface of the heat-exchanging cap and the scalp, a portion of the scalp loses contact with the inner surface of the heat-exchanging cap, leading to problems of the cooling of the scalp becoming uneven or the cooling efficiency deteriorating. As a method of reducing such a gap, in order to facilitate conforming to the shape and size of a user's head, fitting an inner element made of a flexible material between heat-exchanging cap and the scalp is contemplated. However, given that fabricating inner elements from materials such as textiles or foams is the general practice, and that such materials have thermally insulating properties, the presence of the inner element interferes with heat conduction, and a problem that the cooling efficiency ends up being reduced arises instead.

Therein, in consideration of such issues, the applicant in the present application proposed, as published in Japanese Unexamined Pat. Pub. No. 2015-158027, a liner that is fitted onto the inside of a cooling cap for cooling a user's head, the liner including a non-woven fabric layer formed of a nonwoven fabric, the nonwoven fabric layer being a water absorption layer made of a highly water-absorbent plastic fiber material.

The liner presented by the applicant in the present application is provided a superior structure, but as a result of clinical trials, the scalp turned red and mild frostbite was occasionally observed in some users. That is, the liner according to Pat. Pub. No. 2015-158027 is believed to bring about overcooling to the scalp.

A similar problem is noted in article entitled “Managing Hair Loss with Scalp Cooling During Chemotherapy for Solid Tumors” on the website of the Memorial Sloan Kettering Cancer Center, which the website notes is “the world's oldest and largest private cancer center.” The Sloan Kettering article gives the following description of scalp cooling for chemotherapy patients.
Scalp cooling has been used by people in Canada and parts of Europe for years. It has more recently been used in the United States since the Food and Drug Administration (FDA) approved scalp cooling machines in 2015. The FDA approval was based on clinical trials (research studies) that showed what we might expect from scalp cooling and how to best use this method to reduce hair loss during chemotherapy for breast cancer.
( . . . )
Most people can tolerate scalp cooling, but about 30% to 50% of the time people have side effects such as headaches, nausea, . . . scalp pain, or feeling chilly. Some people also find the caps uncomfortably cold. A small number of people decide to stop using scalp cooling because of these side effects.

The website of the aforementioned Paxman company (paxmanscalpcooling.com) provides information for users of the company's “cold cap” system, including tutorial videos under the “Support & Guidance” section of its website, at a sister domain name (coldcap.com/support-guidance/tutorial-videos/). Germane to the present disclosure are the following statements made in the tutorial videos for patient Joe.
Getting a good cap fit before each treatment is the most important thing you can do to ensure the best possible outcome from scalp cooling.
( . . . )
There is a three-point checklist to determine a good fit: One, not too small—make sure that the cap doesn't need to be forced down to fit on the head. This will result in a gap at the crown, as the cap will slowly rise up on the head, particularly as the cap will tighten when it is attached to the system. If there is a gap at the crown, there will be a feeling of movement or bounce if you press down on the cap. It is vitally important that there is contact between the cap and your crown to avoid hair loss. Two, cover the hairline. You need to make sure that the cap covers most if not all of the hairline at the front and back. Please note that sideburns or areas of hair in front of the ears won't be covered due to the shape of the cap. Three, not too big. You can tell if the cap is too big if it can be moved around easily on the head, or if there are large gaps at the back or sides between the scalp and the cap. It is fine to be able to fit a flat hand into the cap at the back of the head, but there shouldn't be space for two hands or a fist. It is important to remember that while a consistent fit all over is important, a cap that is too small can be as detrimental for hair retention as a cap that is too large. (Emphasis added.)

One of the tutorial videos for patient Joe is called “Hints & Tips,” and gives the following advice.
Make sure that your forehead is protected. There are a lot of nerve endings in your forehead. Making sure the inner cap {i.e., heat-exchanging cap constituting the cooling-medium passageway} isn't in direct contact with your scalp helps to really improve tolerability. Headbands don't suit everyone but there are some alternatives that you can use to help protect your forehead or other exposed areas of the scalp. Gauze can work really well for the forehead, and can be cut to the right size. Gauze can be provided by your clinical team. For people with a high hairline or widow's peak, circular cotton wool pads can be very helpful. Fold them in half over a credit card and use the card to slide them up into the cap. If you are experiencing patchy hair loss, there is no reason to stop scalp cooling, but we would always advise that any exposed scalp is protected from direct contact with the cold cap. If possible, you can rearrange any retained hair to cover exposed areas of scalp. Or alternatively, you can use an [operating] theater cap. Just prepare your hair in the normal way, and then put the theater cap on before you fit the inner cap.

Thus, Paxman suggests using gauze, in pieces that must be cut to size, using cotton wool pads folded over a credit card so that they may be slid up between the heat-exchanging cap and the user's head, or even using an operating theater cap. The fact that the company's device is at the forefront of scalp-cooling technology, and yet the company suggests makeshift solutions, such as wearing a theater cap, for “making sure the inner cap isn't in direct contact with [the] scalp . . . to improve tolerability,” indicates that there is a felt need in the industry for a better solution to the problem getting a good scalp-cooling cap fit, and yet ensuring that the cap constituting the cooling-medium passageway is not in direct contact with the patient's scalp.

It is important to note that chemotherapy patients taking advantage of such cooling caps must of course fit the cap on at every treatment session, and that the fit of the cap is of critical importance for scalp cooling to be of maximum effectiveness in preventing hair loss.

Also of noteworthy importance is that the heat-exchanging cap itself is not a hard carapace such as a motorcycle helmet, football helmet, or construction helmet; it is a soft flexible cap design to form-fit the individual patient's scalp, and therefore requires considerable adjustment—a process that can be arduous.

### BRIEF SUMMARY OF THE INVENTION

Therein, an object of the invention is to afford an easy-fitting coif, having a suitable degree of heat-insulating effectiveness, for use beneath the heat-exchanging cap that is the fundamental component of a chemotherapeutic scalp-cooling device or system.

The present invention is a coif—and old-fashioned term availed of by the present disclosure as best characterizing a novel product—worn under, and configured to function in association with, the cooling-fluid circulating cap of a scalp-cooling device or system. The coif is designed to form-fit to the wearer's head with minimal effort on the wearer's part, or on the part of someone assisting the wearer in placing the coif over the wearer's scalp and nape.

The present invention is directed to a coif that facilitates not only its own placement on the patient-wearer's head prior to scalp-and-nape-covering placement of the heat-exchanging cap, but also ensures maximum effectiveness of the cooling process that the scalp-cooling device or system carries out.

An important feature of a coif of the present invention is that it covers the nape. The coif covers beneath where the cooling-medium circulation plumbing (supply and return conduits), encased in in a tubular sheath(s), feeds into and runs out of the heat-exchanging cap.

In an essential aspect of the present invention, a chemotherapeutic coif form-fits to a wearer's scalp and nape by being worn beneath a heat-exchanging cap of a scalp-cooling device. The chemotherapeutic coif is constituted from a fabric body, wherein “fabric” should be understood in the present disclosure as encompassing natural fibers such as cotton, silk, or even paper; synthetic fibers such as polyester or other resin polymer suitable for forming thin cloth; and nonwoven textiles. The fabric body defines inner and outer surfaces, and is made up of: a main body including a frontal region for covering the forehead including the brow, an occipital region for covering the occiput, and a nape region for covering the nape; a left temporal piece and a right temporal piece connected to the frontal region and the occipital region of the main body and extending in respective rear end-ward left and right finlike protrusions, leaving a gap between each finlike protrusion and the nape region. A handle is provided on an edge portion of the frontal region, and with the heat-exchanging cap in place may be tugged on by the wearer to pull out any wrinkles in the coif to eliminate any gap between the coif and the heat-exchanging cap. A hook-and-loop fastener is provided on the inner surface of the finlike protrusion on the left temporal piece, and a hook-and-loop fastener is provided on the inner surface of the finlike protrusion the right temporal piece. Further, a hook-and-loop fastener is provided on each of left and right sides of the outer surface of the nape region to be connectable to the hook-and-loop fastener provided on the inner surface of the finlike protrusion on the left temporal piece and connectable to the hook-and-loop fastener provided on the inner surface of the finlike protrusion the right temporal piece. The hook-and-loop fasteners on the inner surface of the finlike protrusions thus oppose the hook-and-loop fasteners on the outer surface of the nape region across the respective gaps, such that fitting the heat-exchanging cap over the coif on a wearer's head connects the opposing hook-and-loop fasteners to fit the coif to the wearer's head dimensions.

The coif may be constituted essentially of a single-ply material or may be dual layered, constituted by an inner lining of fabric or other suitable material, and an outer shell of fabric or other suitable material.

The chemotherapeutic coif according to the invention can be adapted to persons with different head sizes by adjusting the size of the chemotherapeutic coif or selecting from several kinds of sizes, and a stable wearing state can be obtained. In addition, since a nonwoven or like fabric is used as the material, it is less likely to deteriorate, and there is also little concern of causing itching or rash on the human body. In addition, the medical cap may be made for washing and reuse, or may be made disposable.

From the following detailed description in conjunction with the accompanying drawings, the foregoing and other objects, features, aspects and advantages of the present invention will become readily apparent to those skilled in the art.

### BRIEF DESCRIPTION OF THE SEVERAL VIEWS OF THE DRAWINGS

FIG. 1 is a open-out view of a medical cap involving the invention;

FIG. 2 is a right-side view of the medical cap involving the invention;

FIG. 3A is a front view of the medical cap involving the invention and FIG. 3B is a rear view thereof; and

FIG. 4 is a view illustrating a state in which a user has fitted on the medical cap involving the invention.

### DETAILED DESCRIPTION OF THE INVENTION

Hereinafter, embodiments of the invention will be described in detail with reference to the drawings. In each of the drawings, the same parts are denoted by the same reference numerals and repeated description will not be provided. It should also be noted that the drawings may be exaggerated for the purpose of understanding the invention and are not necessarily drawn to scale. Further, the invention is not limited to the examples illustrated below.

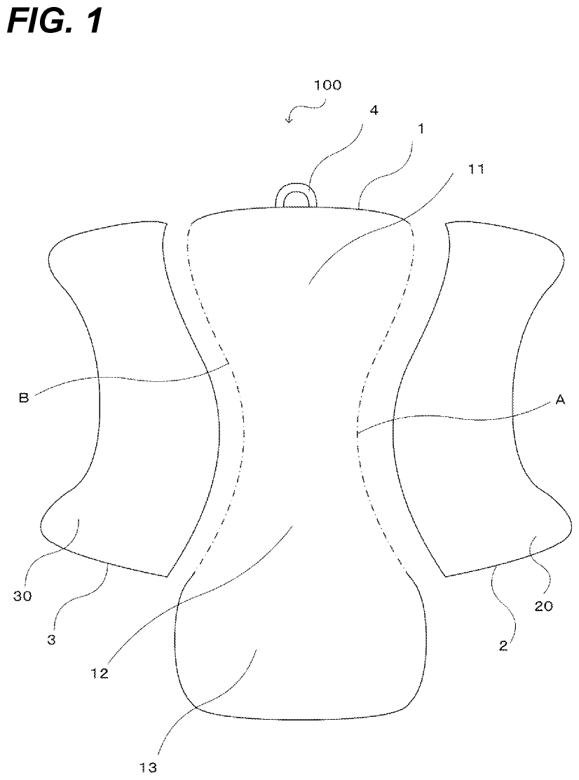

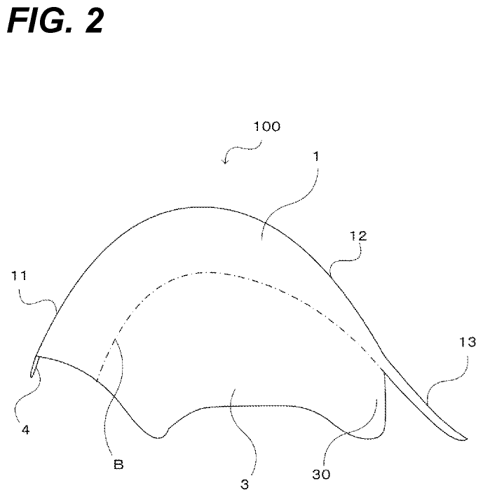

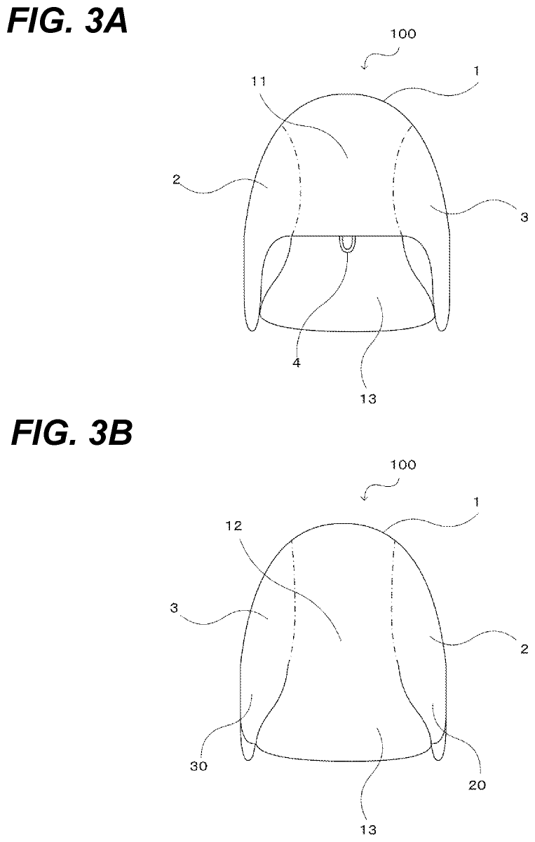

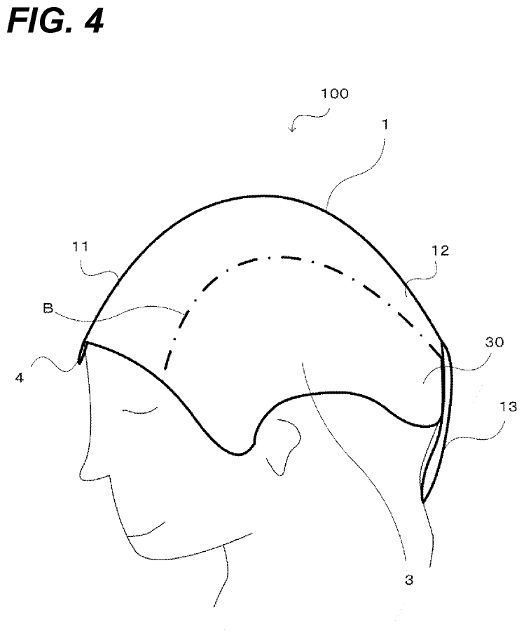

An exemplary embodiment will be described in detail with reference to the drawings. FIG. 1 is an opened-out view of a chemotherapeutic coif, which is constituted by a fabric body defining inner and outer surfaces, according to the invention. FIG. 2 is a right-side view of the chemotherapeutic coif according to the invention. FIG. 3A is a front view of the chemotherapeutic coif according to the invention, and FIG. 3B is a rear view thereof. FIG. 4 is a view illustrating a state in which user has fitted on a chemotherapeutic coif according to the invention. As illustrated in FIGS. 1 to 4, a chemotherapeutic coif 100 according to Example 1 generally includes a main body 1, a left temporal piece 2, and a right temporal piece 3.

Here, referring to FIG. 1: The main body 1 includes a frontal region 11, an occipital region 12, and a nape region 13. The main body 1 is integrally sewn with the left temporal piece 2 at a sewn part A (indicated by alternating long and short dashed lines), and is integrally sewn with the right temporal piece 3 at a sewn part B (indicated by alternating long and short dashed lines appearing in all the figures, with the reference mark “B” appearing in FIGS. 2 and 4 as well in FIG. 1). In addition, connection parts between the main body 1 and the left temporal piece 2 and between the main body 1 and the right temporal piece 3 are not restricted to sewing. The left temporal piece 2 and the right temporal piece 3 respectively form finlike protrusions 20 and 30 at the rear end, and a gap is formed between the finlike protrusions 20 and 30 and the nape region 13.

As illustrated in FIG. 1, the main body 1 may have a handle 4 attached to the front end, that is, an edge portion of the frontal region 11. When a user wears the chemotherapeutic coif 100 and fits on the heat-exchanging cap for a scalp-cooling device, the user can correct displacement of the chemotherapeutic coif 100 by pulling the handle 4.

In addition, the fabric body used for the chemotherapeutic coif 100 may have a suitable degree of heat-insulating effectiveness. That is, any material that can be processed into the shape of the chemotherapeutic coif 100 illustrated in FIGS. 1 to 4 may be used. For example, it is possible to select chemical fibers such as polyester, cotton, nonwoven fabric, and paper as the fabric body, silk, plastics such as thin cloth, and the like as the other material.

Here, FIG. 2 is referred to. FIG. 2 is a right-side view of the chemotherapeutic coif 100 according to the invention. The right temporal piece 3 is sewn to the main body 1 at the upper end. The right temporal piece 3 has the finlike protrusion 30 formed at the rear end, wherein a gap is formed between the finlike protrusion 30 and the nape region 13. A lower line of the right temporal member 3 may be concavely curved so as not to cover the ear. Although not illustrated, a hook-and-loop fastener may be provided on the inner surface of the fabric constituting the finlike protrusion 30 of the right temporal piece 3, and formed to be connectable to a hook-and-loop fastener provided on an opposing portion of the outer surface of the fabric constituting the nape region 13, across the gap from the finlike protrusion 30, to fit the head dimensions of a user. Since the left lateral side is the same as the right lateral side, the description thereof will not be provided.

Here, reference is made to FIG. 3A and 3B. FIG. 3A is a front view of the chemotherapeutic coif 100 according to the invention, and FIG. 3B is a rear view thereof. As illustrated in FIG. 3A, the handle 4 may be attached to the front end of the main body 1, that is, the edge portion of the frontal region 11. The handle 4 may be any member capable of being gripped and pulled by the user, and may be a loop-like member. Alternatively, a tongue piece member may be sewn to the front end of the frontal region 11.

Here, reference is made to FIG. 4. FIG. 4 is a view illustrating a state in which a user has fitted on a chemotherapeutic coif 100 according to the invention. As illustrated in FIG. 4, it will be appreciated that the chemotherapeutic coif 100 according to the invention is a chemotherapeutic coif made up of a fabric body sewn to cover the head of the user, and is constituted from the main body 1 furnished with the frontal region 11 for covering the forehead including the brow, the occipital region 12 for covering covers the occiput, and the nape region 13 for covering the nape, and is constituted from the right temporal piece 3 connected to the main body 1; wherein the right temporal piece 3 connected to the main body 1 forms the finlike protrusion 30 at the rear end, and a gap is formed between the finlike protrusion 30 and the nape region 13. A gap of this sort allows the chemotherapeutic coif to be adapted to people with different head sizes, yielding a stable wearing situation.

The chemotherapeutic coif 100 of the invention is suitable as a liner element worn under the heat-exchanging cap of a scalp cooling device or system, since the coif has a suitable degree of heat-insulating effectiveness, and when the heat-exchanging cap is fitted on with the liner element being worn, since the liner element serves as a cushioning material between the heat-exchanging cap and the scalp, a comfortable fit is provided for the user.

While the preferred embodiment of the chemotherapeutic coif according to the invention has been illustrated and described, it will be understood that various modifications can be made without departing from the scope of the invention.

A chemotherapeutic coif involving the invention finds broad utility not only as a liner element of a heat-exchanging cap for a scalp cooling device, but also as a hat that covers the head in cases where a patient has lost hair due to the side effects of anticancer drugs.

### LEGEND

100: coif
1: main body
11: frontal region
12: occipital region
13: nape region
2: left temporal piece
3: right temporal piece
20, 30: fin-like protrusion
4: handle

Only selected embodiments have been chosen to illustrate the present invention. To those skilled in the art, however, it will be apparent from the foregoing disclosure that various changes and modifications can be made herein without departing from the scope of the invention as defined in the appended claims. Furthermore, the foregoing description of the embodiments according to the present invention is provided for illustration only, and not for limiting the invention as defined by the appended claims and their equivalents.

What is claimed is:

1. A chemotherapeutic coif form-fitting to a wearer's scalp and nape by being worn beneath a heat-exchanging cap of a scalp-cooling device, the chemotherapeutic coif comprising:
a fabric body defining inner and outer surfaces, the fabric body made up of
a main body including a frontal region for covering the forehead including the brow, an occipital region for covering the occiput, and a nape region for covering the nape,
a left temporal piece and a right temporal piece connected to the frontal region and the occipital region of the main body and extending in respective rear end-ward left and right finlike protrusions, leaving a gap between each finlike protrusion and the nape region;
a handle on an edge portion of the frontal region;
a hook-and-loop fastener provided on the inner surface of the finlike protrusion on the left temporal piece, and a hook-and-loop fastener provided on the inner surface of the finlike protrusion the right temporal piece; and
a hook-and-loop fastener provided on each of left and right sides of the outer surface of the nape region to be connectable to the hook-and-loop fastener provided on the inner surface of the finlike protrusion on the left temporal piece and connectable to the hook-and-loop fastener provided on the inner surface of the finlike protrusion the right temporal piece; wherein
the hook-and-loop fasteners on the inner surface of the finlike protrusions oppose the hook-and-loop fasteners on the outer surface of the nape region across the respective gaps, such that fitting the heat-exchanging cap over the coif on a wearer's head connects the opposing hook-and-loop fasteners to fit the coif to the wearer's head dimensions.

2. A scalp cooling system for cooling the human scalp, comprising:
a heat-exchanging cap; and
the chemotherapeutic coif according to claim 1.
